(19) United States
(12) Patent Application Publication
(10) Pub. No.: US 2020/0060548 A1
(43) Pub. Date: Feb. 27, 2020

(54) Diagnostic Device Using Near Infrared Light

(71) Applicant: Signal Ophthalmic Consulting, LLC, Memphis, TN (US)

(72) Inventors: Ramon Gomez, Memphis, TN (US); Whitney H. Hauser, Memphis, TN (US)

(21) Appl. No.: 16/108,878

(22) Filed: Aug. 22, 2018

(51) Int. Cl. A61B 5/00; A61B 3/14

(52) U.S. Cl. CPC A61B 5/0075; A61B 5/0086; A61B 5/1455; A61B 3/145; A61B 5/0064

(57) ABSTRACT

Disclosed is a diagnostic tool configured to evaluate human tissue and associated structures. The device can generate electromagnetic radiation (EMR) at or near a near infrared (NIR) window, the NIR window being a predetermined window that includes a range of wavelengths that allow for maximum penetration depth of the EMR being emitted from the device through tissue. The EMR emitted by the device can form a uniform, diffuse NIR transmission corresponding to the NIR window. A controller can be used to control current supply to the light source so as to modulate the intensity or amplitude of the EMR.

### FIELD OF THE INVENTION

Embodiments of the invention relate to a diagnostic device that can generate a uniform, diffuse near infrared (NIR) transmission spanning a NIR therapeutic window.

### BACKGROUND OF THE INVENTION

Conventional methods of evaluating eyelids and other tissue are limited to gross observation by illumination of the eyelid with diffuse white light. (See FIG. 1). Conventional approaches provide an observational perspective only. No or very limited photographic recording or analysis is applied. Photographic recording of observations is limited due to dexterity limitations (e.g., the application of a white light instrument, manipulation and focusing of a biomicroscope, and capture of a photograph). The white light application provides a gross visualization without distinct detail necessary for medical review and longitudinal analysis for pathologies identified.

Some tissue observation techniques can utilize near-infrared spectroscopy (NIRS). NIRS is a method by which the interaction between matter and electromagnetic radiation (EMR) is studied. The near-infrared window, also known as the optical or therapeutic window, is defined as wavelengths between 650 nm to 2500 nm. Use of radiation having wavelengths within the NIR therapeutic window during NIRS allows for maximum penetration depth of light through living tissue, which can be beneficial for diagnostic purposes. The NIR therapeutic window may be categorized into: a first window (650-950 nm), a second window (1100-1350 nm), and a third window (1600-1870 nm).

Known diagnostic devices and other devices utilizing NIRS are limited in functionality. One of the reasons for this can be that they use specific lights sources that are dedicated for a specific purpose and a specific type of tissue. These devices generate a single wavelength or a very narrow wavelength band to perform the NIRS. This hinders versatility by limiting penetration into the tissue. In addition, this can result in poor visualization for some tissue structures. Known diagnostic devices and near infrared light generators and analyzers can be appreciated from U.S. Pat. Nos. 7,029,439, 7,214,952, 7,344,272, 7,540,613, 7,568,802, 7,657,292, 7,712,901, 8,255,039, and 8,944,596.

### SUMMARY OF THE INVENTION

Embodiments of the device can be a diagnostic tool configured to evaluate human tissue and associated structures. The device can generate electromagnetic radiation (EMR) at or near a near infrared (NIR) window, the NIR window being a predetermined window that includes a range of wavelengths that allow for maximum penetration depth of the EMR being emitted from the device through tissue. In some embodiments, the EMR emitted by the device forms a uniform, diffuse NIR transmission corresponding to the NIR window. In some embodiments, a controller can be used to control current supply to the light source so as to modulate the intensity or amplitude of the EMR.

Illumination of the surface of the eyelids by direct contact can provide a more detailed view of the anatomical appearance and physiological function. In addition, written descriptions or external ocular photography can be performed to record structural findings with techniques that employ direct contact. The eyelids may be directly illuminated by applying a uniform, diffuse light to the lid structure. The external application of the light to the lid provides a localized observable area. (See FIGS. 3A-3B).

In at least one embodiment, a diagnostic tool can include at least one light source configured to generate electromagnetic radiation (EMR) within a range from 650 nm to 2500 nm, the EMR being emitted through a light emission region. The diagnostic tool can include a detector unit configured to detect interactions of the EMR with tissue. In some embodiments, the light emission region is configured as a diffuser to cause the EMR to be a uniform, diffuse electromagnetic transmission spanning the range from 650 nm to 2500 nm or an intermediate range within the range from 650 nm to 2500 nm. In some embodiments, the light emission region is configured to cause the EMR to be a uniform, diffuse electromagnetic transmission spanning a range from 650 nm to 1870 nm or an intermediate range within the range from 650 nm to 1870 nm.

In some embodiments, the at least one light source includes a light emitting diode (LED). In some embodiments, the at least one light source includes a plurality of light sources. In some embodiments, the plurality of light sources includes: a first light source configured to generate EMR within a first predetermined band of wavelengths; a second light source configured to generate EMR within a second predetermined band of wavelengths; and a third light source configured to generate EMR within a third predetermined band of wavelengths.

In some embodiments, the first predetermined band of wavelengths is different from the second predetermined band of wavelengths and the third predetermined band of wavelengths; the second predetermined band of wavelengths is different from the first predetermined band of wavelengths and the third predetermined band of wavelengths; and the third predetermined band of wavelengths is different from the second predetermined band of wavelengths and the first predetermined band of wavelengths.

In some embodiments, the first predetermined band of wavelengths is less than the second predetermined band of wavelengths; and the second predetermined band of wavelengths is less than the third predetermined band of wavelengths.

In some embodiments, the first predetermined band of wavelengths is within a range from 650 nm to 950 nm; the second predetermined band of wavelengths is within a range from 1100 nm to 1350 nm; the third predetermined band of wavelengths is within a range from 1600 nm to 1870 nm; and the light emission region is configured to cause the EMR to be a uniform, diffuse electromagnetic transmission spanning a range from 650 nm to 1870 nm or an intermediate range within the range from 650 nm to 1870 nm.

In some embodiments, the light emission region includes a lens made from glass, plastic, polymer, ceramic, and/or epoxy. In some embodiments, lens is configured as a diffuser.

Some embodiments include a controller configured to control the intensity of the EMR being emitted from the at least one light source. In some embodiments, the controller is a variable resistor rheostat. In some embodiments, the controller is configured to control the intensity of the EMR being emitted from each light source individually. In some embodiments, the controller is configured to control the intensity of the EMR being emitted from each light source in unison.

In at least one embodiment, a diagnostic tool can include a housing configured as a hand-held unit for placement adjacent tissue to be analyzed via spectroscopy. The diagnostic tool can include a light emission region formed in a portion of the housing. The diagnostic tool can include at least one light source configured to generate electromagnetic radiation (EMR) within a range from 650 nm to 2500 nm, the EMR being emitted through the light emission region. The diagnostic tool can include a detector unit configured to detect interactions of the EMR with the tissue. The diagnostic tool can include a power source configured to facilitate transfer of electrical current or electrical voltage from an electrical power supply. In some embodiments, the light emission region is configured as a diffuser to cause the EMR to be a uniform, diffuse electromagnetic transmission spanning the range from 650 nm to 2500 nm or an intermediate range within the range from 650 nm to 2500 nm. In some embodiments, the power supply is a battery unit.

In at least one embodiment a diagnostic system can include a diagnostic tool. The diagnostic tool can include at least one light source configured to generate electromagnetic radiation (EMR) within a range from 650 nm to 2500 nm, the EMR being emitted through a light emission region. The diagnostic tool can include a detector unit configured to detect interactions of the EMR with tissue and generate detection data. In some embodiments, the light emission region is configured as a diffuser to cause the EMR to be a uniform, diffuse electromagnetic transmission spanning the range from 650 nm to 2500 nm or an intermediate range within the range from 650 nm to 2500 nm. The diagnostic system can include a computer in communication with the diagnostic device. The computer can be configured to receive the detection data and generate an image and/or a video that is representative of the detection data.

In some embodiments, the computer is configured to analyze the detection data via spectroscopy. In some embodiments, the computer is configured to generate a user interface programmed to facilitate image or video processing, enhancing, analysis, and/or storage of the image and/or the video. In some embodiments, the computer is configured to generate text superimposed over the image and/or the video, the text comprising dimensions of the tissue and/or structures associated with the tissue.

In at least one embodiment, a method of examining eyelid tissue can involve placing a light source adjacent an eyelid and applying pressure to the eyelid via exertion of pressure to the eyelid through a movement of the light source, the exertion of pressure causing the eyelid to be everted over at least a portion of the light source. The method can involve generating electromagnetic radiation (EMR) within a range from 650 nm to 2500 nm, the EMR being a uniform, diffuse electromagnetic transmission spanning the range from 650 nm to 2500 nm or an intermediate range within the range from 650 nm to 2500 nm. The method can involve detecting interactions of the EMR with tissue and associated structures of the eyelid.

In some embodiments, the tissue and associated structures of the eyelid include meibomian glands. Some embodiments can involve identifying morphologic structures of the tissue and associated structures of the eyelid. Some embodiments can involve identifying morphological changes in the tissue and associated structures of the eyelid. Some embodiments can involve generating an image and/or a video of the tissue and associated structures of the eyelid. Some embodiments can involve recording morphologic structures of the tissue and associated structures of the eyelid via the image and/or the video. Some embodiments can involve recording morphological changes in the tissue and associated structures of the eyelid via the image and/or the video.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present invention will be more apparent from the following more particular description thereof, presented in conjunction with the following drawings. Like reference numbers used in the drawings may identify like components.

FIG. 1 shows a prior art illumination technique of observing the eyelid via non-direct contact of a light source being used to illuminate the eye.

FIG. 2 shows an embodiment of a diagnostic device.

FIGS. 3A-3B show exemplary uses of an embodiment of a diagnostic device.

FIG. 4 shows an exemplary uniform, diffuse radiation pattern of electromagnetic radiation that can be generated from an embodiment of a diagnostic device.

FIG. 5 shows an exemplary communication network topology that can be used to with an embodiment of a diagnostic system.

FIG. 6 shows an exemplary user interface that can be used with an embodiment of a diagnostic system.

FIGS. 7-9 show exemplary user interfaces that can be used to perform image and/or video processing for an embodiment of a diagnostic system.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

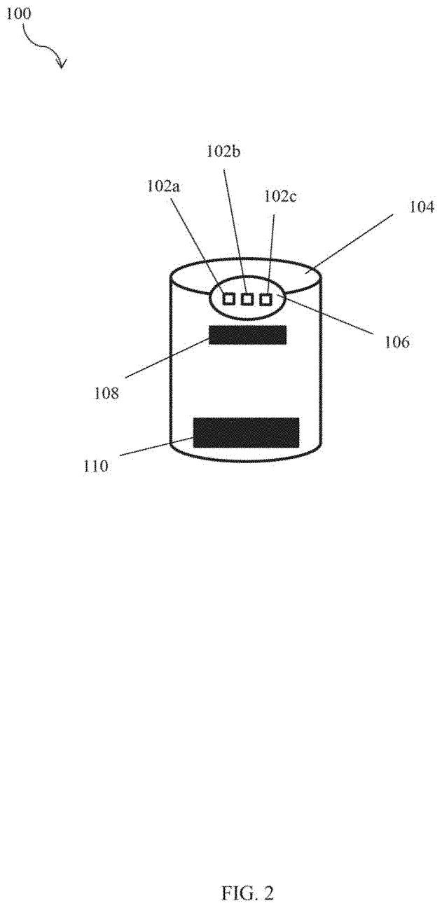

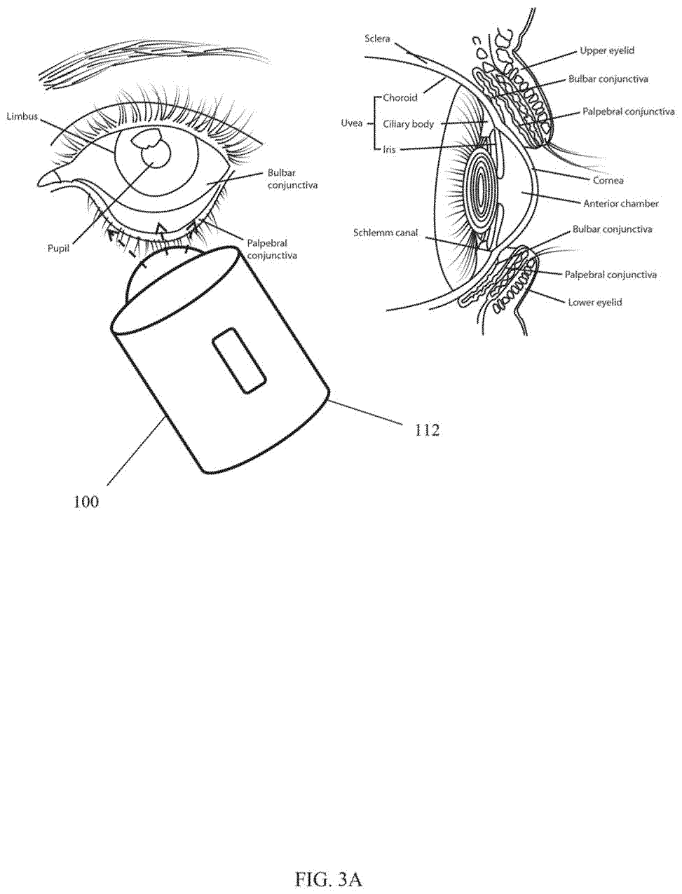

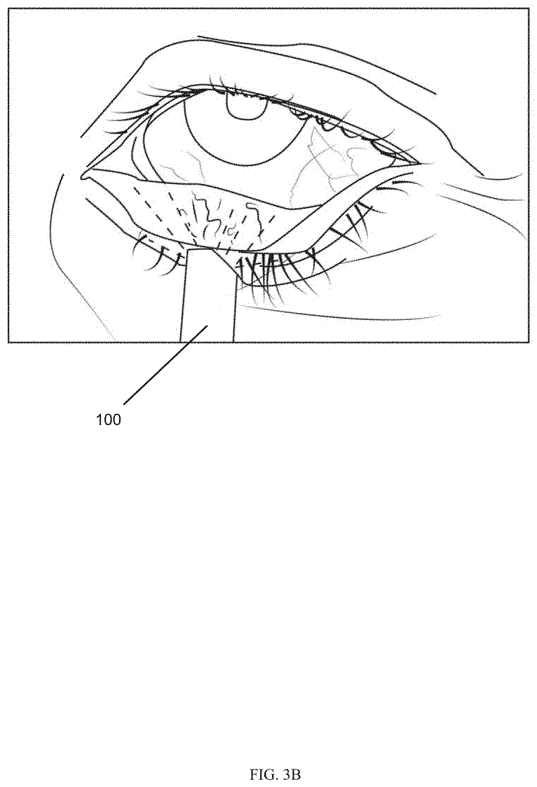

Referring to FIGS. 2-4, embodiments of the device 100 can relate to a diagnostic device configured to evaluate animal tissue and associated structures. The tissue can be human tissue. The tissue can be living tissue or dead tissue. The tissue can be epithelial tissue, connective tissue, muscle tissue, nervous tissue, etc. The associated structures can be blood vessels, glands, lesions, etc. (See FIGS. 3A-3B). Exemplary uses of the device 100 describe and illustrate the device 100 being used for diagnosis of tissue and other structures of a human eyelid, which can include the upper eyelid and/or the lower eyelid.

In at least one embodiment, the device 100 is a handheld, battery powered eye diagnostic device that can generate electromagnetic radiation (EMR) at or near a near infrared (NIR) window. The NIR window can be a predetermined window that includes a range of wavelengths. The range of wavelengths can be defined as an optical window or a therapeutic window of wavelengths. This can be wavelengths within a range from 650 nm to 2500 nm. Embodiments of the device 100 can be configured to generate EMR having wavelengths within the NIR window so as maximize penetration of the EMR being emitted from the device 100 through tissue. In some embodiments, the device 100 can have a light source 102 configured to generate EMR with wavelengths that span the NIR window. In a least one embodiment, the light source 102 can have a plurality of light sources 102 located within an epoxy resin so that the EMR emitted by the device 100 forms a uniform, diffuse NIR transmission corresponding to the NIR window. In some embodiments, a controller 112 can be used to control current supply to the light source 102 so as to module the intensity or amplitude of the EMR.

Embodiments of the device 100 can include a housing 104 with necessary components (e.g., circuitry, switches, sensors, processors, etc.) to facilitate controlled operation of at least one light source 102. The housing 104 can be configured as a handheld unit with a light emission region 106. During use of the device 100, the light emission region 106 can be placed adjacent tissue to expose the tissue to EMR. This can include causing EMR to be incident on the tissue, transmitted through the tissue, etc. Interactions of the EMR with the tissue can be detected, recorded, and processed. This can be done in a manner to facilitate spectroscopy, which can include near-infrared spectroscopy (NIRS). Using NIRS techniques on the tissue can allow a user to perform diagnostics on the tissue and associated structures and make assessments therefrom.

Embodiments of the device 100 can include at least one light source 102. The light source 102 can be configured to generate the EMR that is caused to emanate from the light emission region 106. The light source 102 can be a light emitting diode (LED), an organic LED, an active-matrix organic LED, incandescent bulbs, halogen bulbs, etc. The light source 102 can be configured to generate EMR that is within a predetermined band of wavelengths. The predetermined band of wavelengths can be defined as a wavelength range. The wavelength range can be within the infrared spectrum. For example, the wavelengths within a predetermined band of wavelengths 103 can be within a range from 780 nm to 2500 nm, or any intermediate range within that range. In some embodiments, wavelength range can be within the near infrared spectrum. For example, the wavelengths within a predetermined band of wavelengths 103 can be within a range from 650 nm to 2500 nm, or any intermediate range within that range.

Some embodiments can include a plurality of light sources 102. For example, the device 100 can have a first light source 102a, a second light source 102b, a third light source 102c, etc. The first light source 102a can be configured to generate EMR within a first predetermined band of wavelengths 103a. The second light source 102b can be configured to generate EMR within a second predetermined band of wavelengths 103b. The third light source 102c can be configured to generate EMR within a third predetermined band of wavelengths 103c. The first predetermined band of wavelengths 103a, or at least a portion thereof, can be the same as or different from the second predetermined band of wavelengths 103b and/or the third predetermined band of wavelengths 103c. The second predetermined band of wavelengths 103b, or at least a portion thereof, can be the same as or different from the first predetermined band of wavelengths 103a and/or the third predetermined band of wavelengths 103c. The third predetermined band of wavelengths 103c, or at least a portion thereof, can be the same as or different from the second predetermined band of wavelengths 103b and/or the first predetermined band of wavelengths 103a.

In some embodiments, the first predetermined band of wavelengths 103a, or at least a portion thereof, can be less than the second predetermined band of wavelengths 103b. The second predetermined band of wavelengths 103b, or at least a portion thereof, can be less than the third predetermined band of wavelengths 103c. In at least one embodiment, the first predetermined band of wavelengths 103a can be within a range from 650 nm to 950 nm. The second predetermined band of wavelengths 103b can be within a range from 1100 nm to 1350 nm. The third predetermined band of wavelengths 103c can be within a range from 1600 nm to 1870 nm.

Embodiments of the device 100 can include a detector unit 108. The detector unit 108 can be configured to receive EMR after it has been exposed to the tissue, receive EMR that has been transmitted through the tissue, receive EMR that has been reflected from the tissue, and/or receive EMR that is generated as a result of the interactions of the EMR with the tissue. The detector unit 108 can include optical elements (e.g., lenses, prisms, diffraction gratings, beam splitters, interferometers, etc.), sensors (e.g., light sensor, infrared camera, a charged coupled device, photodiode array, etc.), and a processor (e.g., an integrated circuit, central processing unit, microprocessor, core processor, etc.) configured to receive EMR and identify different wavelengths and/or intensities of the received EMR. For example, the optical elements can be configured to focus the EMR, disperse the EMR, collimate the EMR, filter the EMR, direct the EMR in a certain direction, etc. The sensors can be configured to identify EMR having predetermined intensities, wavelengths, phase, polarization, etc. The processor can be configured to perform various signal processing functions (e.g., as Fourier transforms, Gabor transforms, etc.) to generate data and signal profiles to be further processed and analyzed.

Embodiments of the device 100 can include a power source 110. The power source 110 can be a power outlet (e.g., power cord socket, USB port, etc.) configured to connect the device 100 to an electrical power supply (e.g., 120 VAC electrical supply). Electrical current or electrical voltage can be transferred from the electrical power supply to the components of the device 100 via the power source 110. In some embodiments, the power source 110 can be a battery unit (e.g., 3-Volt lithium battery). The battery unit can be a permanent or replaceable battery unit. The battery unit can be part of or attached to the housing 104. In some embodiments, the power source 110 can include the power outlet and the battery. The power outlet can be configured to transfer electrical current or electrical voltage from the electrical power supply to the components of the device 100 and/or the battery (e.g., recharge the battery). In addition, or in the alternative, the battery unit can be detached from the housing 104 and be recharged at a charging station.

Embodiments of the device 100 can have a light emission region 106. The light emission region 106 can be a portion of the device 100 that holds the light source 102. Embodiments of the light emission region 106 can include a lens configured to allow EMR from the light source 102 to radiate from the device 100. The lens can be glass, plastic, polymer, ceramic, epoxy etc. The lens can be transparent or translucent to the predetermined wavelength range, or at least a portion thereof, of at least one of the light sources 102. In some embodiments of the light emission region 106, the lens can be configured to serve as a focusing element, filter element, a prism element, a reflector element, a refractor element, a diffraction element, a diffuser element, etc.

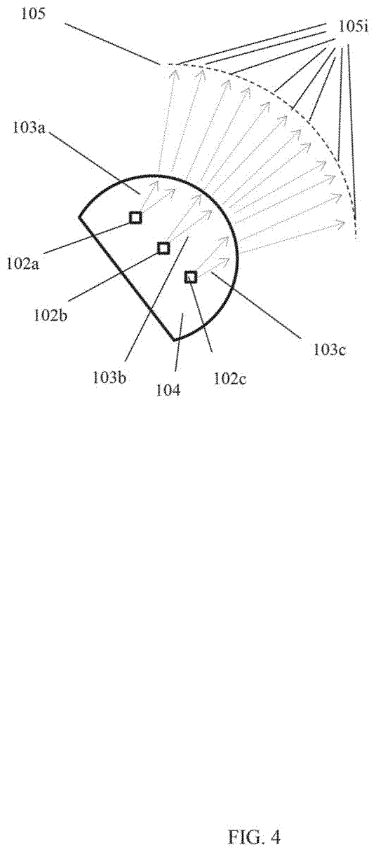

In at least one embodiment, the light emission region 106 can include a lens that is configured as a diffuser. This can be done to generate a uniform, diffuse NIR transmission corresponding to the NIR therapeutic window. For example, the device 100 can be configured to generate EMR with a NIR therapeutic window that is defined as being within a range from 650 nm to 1870 nm. The device 100 can have a first light source 102a configured to generate EMR within a first predetermined band of wavelengths 103a that is from 650 nm to 950 nm. The device 100 can have a second light source 102b configured to generate EMR within a second predetermined band of wavelengths 103b that is from 1100 nm to 1350 nm. The device 100 can have a third light source 102c configured to generate EMR within a third predetermined band of wavelengths 103c that is from 1600 nm to 1870 nm. Each of the first light source 102a, the second light source 102b, and the third light source 102c can be configured to emit their respective EMR through the light emission region 106. The light emission region 106, being configured as a diffuser, can cause the EMR from the three light sources 102 to scatter into a diffuse transmission. The resultant diffuse transmission can be a uniform, diffuse NIR electromagnetic transmission corresponding to the NIR therapeutic window. This can include a uniform, diffuse transmission of EMR having wavelengths that span the range from 650 nm to 1870 nm. (See FIG. 4). A uniform transmission is defined as producing a wavefront profile 105 that has a same (or within a predetermined variance) intensity across the wavefront profile 105. For example, the amplitude or the intensity of each of the light beams forming the wavefront profile 105 is the same or within a narrow range (e.g., within +/−1%, within +/−2%, within +/−3%, etc.) at each point 105i of the wavefront profile 105. Thus, in use, the device 100 can be used to generate a uniformly intense radiation of light to be emitted across the eyelid (e.g., from canthus to canthus).

Embodiments of the device 100 can include a controller 112. The controller 112 can include switches and other control modules to facilitate controlled operation of the various components of the device 100. For example, the controller 112 can be configured to control flow of electrical current from the power source 110 to the light source 102. In some embodiments, the controller 112 can include a rheostat. The rheostat can be a variable resistor rheostat. With embodiments of the controller 112 being configured as a variable resistor rheostat, increasing or decreasing the resistance of the rheostat can result in an increase or a decrease of the electrical current being supplied to the light source 102. This can facilitate control of the intensity of the EMR being emitted from the light source 102. The controller 112 can be configured to control the intensity of EMR from each light source 102 individually, all-together (e.g., in unison), or some individually with some being controlled in unison.

As noted herein, embodiments of the device 100 can be used as a diagnostic tool by performing NIRS on tissue. With the large homogeneity of skin in terms of thickness, structure (presence/absence of blood vessels, sweat/oil glands, lesions), and fat content, it can be beneficial to have a diagnostic device 100 that allows for varying the intensity of the EMR. Varying the intensity of the EMR can enhance and even maximize observation and image processing for diagnostic purposes. In addition to structural differences, NIR spectral ranges can be dependent on chromophores in the skin including: water content, vasodilation, the composition of blood (concentration of hemoglobin, oxygenation status), melanin, etc., and thus being able to vary the intensity of the EMR can further enhance observations and image processing.

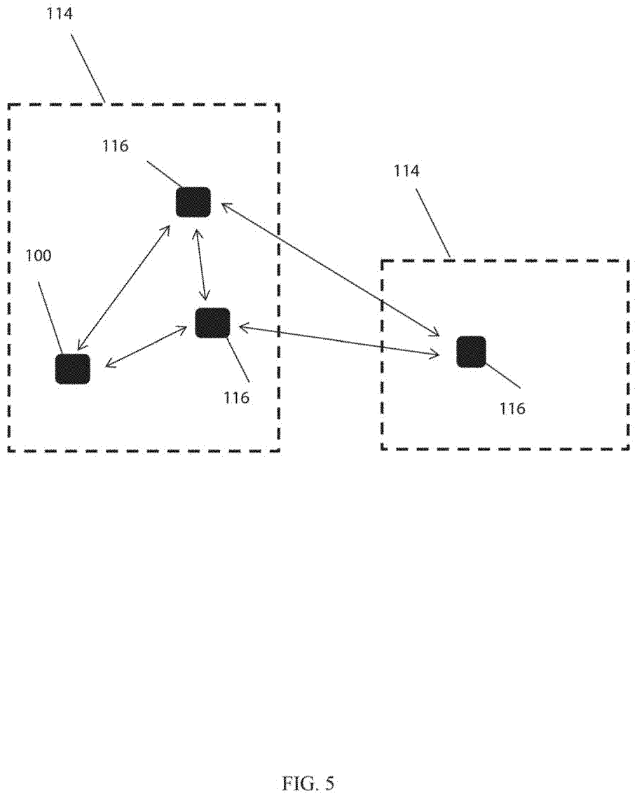

Referring to FIG. 5, in some embodiments, the device 100 can be part of or in connection with a communications network 114. For example, the device 100 can include switches, transmitters, transceivers, routers, gateways, etc. to facilitate communications via a communication protocol that facilitates controlled and coordinated signal transmission and processing. The communication links can be established by communication protocols that allow devices 100 to form the communications network 114 with another device 100 or another apparatus. For instance, the device 100 can be configured to communicate with another device 100 and to facilitate data transmissions to and from devices 100 (or other nodes) within or between discrete communication networks. The communications network 114 can be a long range wired or a wireless network, such as an Ethernet, telephone, Wi-Fi, Bluetooth, wireless protocol, cellular, satellite network, cloud computing network, etc. Embodiments of the communications network 114 can be configured as a predetermined network topology. This can include a mesh network topology, a point-to-point network topology, a ring (or peer-to-peer) network topology, a star (point-to-multiple) network topology, or any combination thereof.

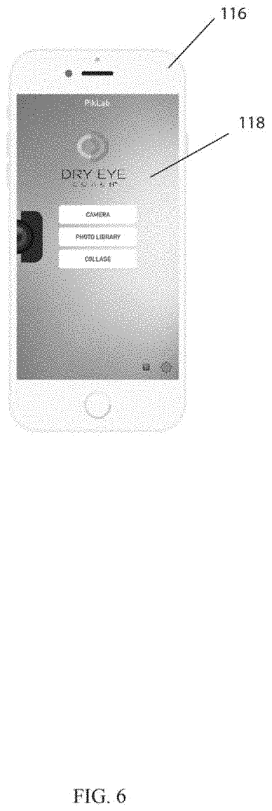

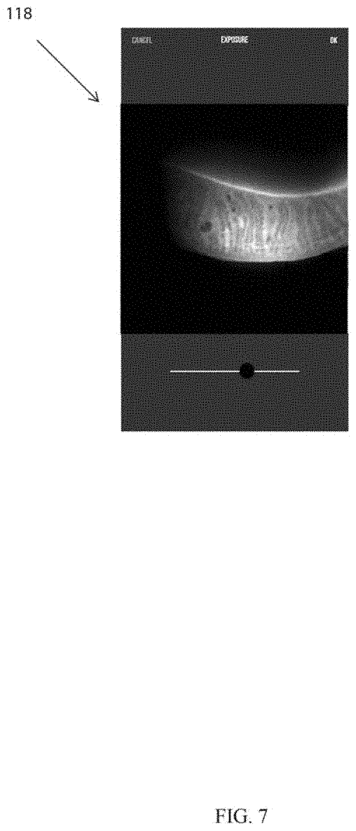

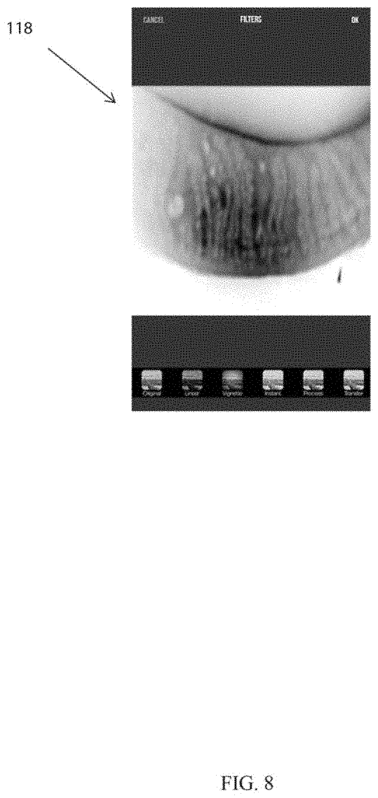

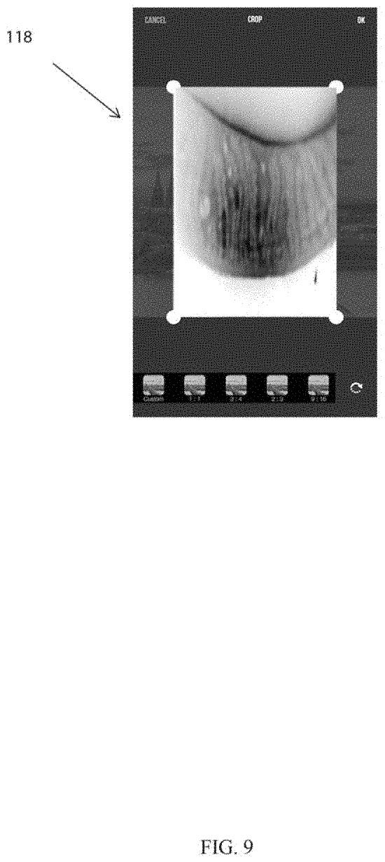

In addition, any of the devices 100 can have an application programming interface (API) and/or other interfaces configured to facilitate a computer 116 that is in communication with the device 100 executing commands and controlling aspects of the device 100. For example, some embodiments of the communications network 114 can include a computer 116 (e.g., a server, a mainframe computer, a desk top computer, a laptop computer, a tablet, a smartphone, etc.) configured to be in communication with the device 100. Embodiments of the computer 116 can be programmed to generate a user interface 118 configured to facilitate control of and display of various operational aspects of image processing and diagnostic features. (See FIGS. 6-9). Aspects of the user interface 118 can also be configured to allow a user to control operational aspects of the device 100.

In at least one embodiment, the device 100 can be communicatively associated with the computer 116. This can be via the communications network 114 and/or via a hardwire connection (e.g., a USB port connection). The EMR received by the detector unit 108 can be converted into detection data via the processor of the device 100. The processor of the device 100 can transmit the detection data to the computer 116. Image processing software and image generating software can be installed on the computer 116 and be executed by the computer 116 to generate an image and/or video that is representative of the detection data. The user interface 118 can be programmed to provide various actionable features and graphical user interfaces (GUIs) to allow a user to control various operational aspects of the computer 116 and/or the device 100. This can include controlling the controller 112 of the device 100, the image processing and image generating aspects of the computer 116, etc.

FIGS. 6-9 show a non-limiting, exemplary implementation of a user interface(s) 118 that can be used. While embodiments of the user interface 118 disclosed herein relate to a computer 116 that is a mobile electronic device, it is understood that the user interface 118 can be configured for a desktop type computer as well. For example, a user interface 118 for a mobile electronic device can be configured to operate on a smaller screen display, with less computational resources, with touchscreen graphics, etc. A user interface 118 for a desktop type computer can be more robust, operate on a larger screen display, and may not have touchscreen graphics.

The exemplary user interface 118 can be a mobile-based interactive user interface 118 for image and/or video processing, enhancing, analysis, and/or storage. The user interface 118 can have a GUI that allows a user to cause the computer 116 to generate an image and/or video that is representative of the detection data from tissue. The GUI can also be configured to display the image and/or video. The image and/or video can be a rendition of the actual tissue or some other graphical or pictorial illustration. The image and/or video can be processed immediately or stored for later processing. If stored, the GUI can allow the user to retrieve the image and/or video from a library of stored images and videos for comparison and/or processing. Storing images and videos can involve sending data representative of the image or video to a non-volatile, non-transitory memory (e.g., a RAMS, a DRAM, a database, etc.).

Additional actionable features of the user interface 118 can be programmed to allow a user to enhance the image and/or video (e.g., improved visibility of objects and structures within the image and/or video). This can be done via various filtering techniques, Gabor processing, pattern recognition, etc.

Additional actionable features of the user interface 118 can be programmed to allow a user to identify and obtain measurements of structures found within the image and/or video. The measurements can be used to determine dimensions and locations of structures (e.g., blood vessels, sweat/oil glands, lesions, fat, etc.) within the image and/or video. In some embodiments, the measurements can be obtained via actuating an actionable feature. In addition, or in the alternative, the measurements can be generated automatically via program logic. In some embodiments, come measurements can be obtained via actuating actionable features while others can be generated automatically.

In some embodiments, the user interface 118 can be programmed to populate a menu that will allow the user to generate an image and/or video from detection data being collected by the device 100 or select an image and/or video from the list of images or videos already stored in the existing library. After the image and/or video is generated or selected, it can be designated for processing. This can be done by selecting actionable features of the menu. These actionable features can allow for editing options and image or video enhancing options (e.g., photo/video adjustment tools, brightness, contrast, saturation, exposure, light balance, inverted color selection, RGB balance, crop and selection with pre-determined settings as well as free-hand cropping tool, tone curve, etc.).

Other image and/or video processing and analysis can be done, which can be initiated automatically (via program logic) by the computer 116 or initiated by a user actuating an actionable feature of the user interface 118. This can include identifying structures within the image and/or video, analyzing the structures, identifying the relative location of the structure, measuring the dimensions of the structures, etc.

The dimensions generated can be expressed in metric (e.g., millimeters) or English standard units (e.g., inches). In some embodiments, measurements of tissue or an associated structure can correspond to the dimensions of the tissue or associated structure within the image and/or r video. In some embodiments, measurements of tissue or an associated structure can correspond to the percentage of area occupied by the tissue or associated structure in relation to the dimensions of the total area of the image and/or video (e.g., expressed as a percentage or a ratio).

Any of the images and/or videos, either before or after being processed, can be transmitted from one computer 116 to another computer 116 within the communications network 114 or to a computer 116 that is on another communication network but is able to connect to the communications network 114. In addition, any of the detection data, before or after being processed, can be transmitted from one device 100 to another device 100 or to a computer 116 within the communications network 114 or to a device 100 or computer 116 that is on another communication network but is able to connect to the communications network 114.

It is contemplated for the user interface 118 to be implemented via application software made available to iOS operating devices, Android devices, etc. It is further contemplated for the software application to be written in X-code programming language. This can allow for the computer 116 (e.g., the smartphone) to perform most of the functions related to taking pictures or videos, storage and sharing of files to external locations, etc. In some embodiments, image and video processing can be provided by the Open CV library distributed for a particular mobile development platform.

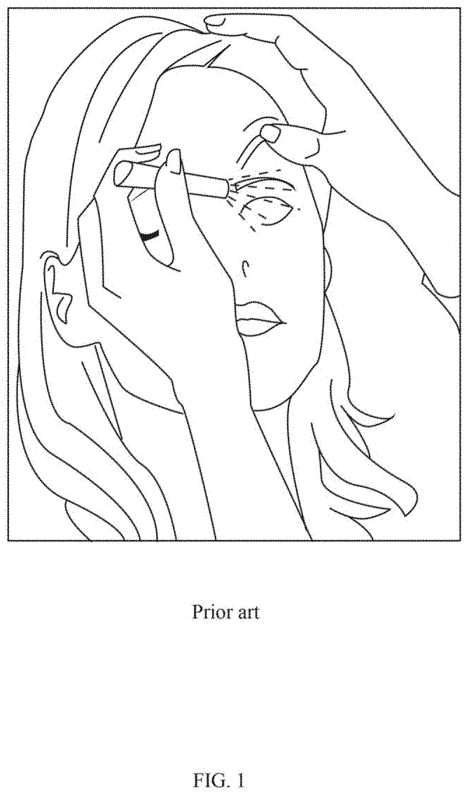

In an exemplary implementation, the device 100 can be positioned so that the light emission region 106 is adjacent the external lower eyelid at the margin or lash line. (See FIGS. 3A-3B). Pressure can be exerted via the device 100 at an upward angle to flip the lower eyelid, causing it to be everted. This can cause the internal palpebral conjunctiva to be exposed while the light emission region 106 maintains contact with the lower eyelid. It should be noted that prior art devices and methods cannot be used so as to be placed adjacent the eyelid. (See FIG. 1). Prior art devices also cannot be used to exert pressure to flip the eyelid and expose the internal palperbral conjunctiva. In some embodiments, a biomicroscope (e.g., a slit lamp) can be used to shine light into the eye. This can be done while the device 100 is being used. The controller 112 can be activated to cause the light source 102 to emit EMR. The device 100 may be repositioned as needed to maximize exposure of the EMR from o the lower eyelid from the device 100. The controller 112 can be adjusted to find the ideal intensity of the NIR light source to visualize the lower eyelid tissue and associated structures. For example, after the diffuse illumination of radiation is caused to be incident upon the tissue, the controller 112 can be adjusted to cause the intensity to be adjusted. As the intensity is adjusted, a user can visually inspect (or the computer 116 can perform image analytics) to determine the best intensity that generates the best image and/or video of the tissue and/or associated structures.

Detection data can be collected related to eye conditions such as retention cysts, sebaceous gland obstruction/atrophy, malignant/benign lid lesions (including but not limited to chalazia, hordeola, etc.), etc. The detection data can be transmitted to the computer device 116 for further processing. This can include generating images and/or videos of the detection data, storing the detection data and/or the images and/or videos, manipulating the detection data, enhancing the images and/or videos, etc. The detection data and/or the images and/or video can be observed via the display of the computer 116. This can be done in real-time or at a later time. Identification of tissue and associated structures, as well as the dimensions of each can be determined and displayed via the display of the computer 116. The identified tissue and associate structures and the dimensions of the same can be displayed as superimposed text on the image and/or video.

A non-limiting, exemplary implementation of the device 100 can be evaluation of meibomian glands located at the rim of the eyelid. Illumination of the eyelid with the device 100 can reveal the vascular and gland structure of the eyelid, including those of the meibomian glands. In addition, images and/or videos of the vascular and gland structure can be generated and displayed via the computer 116. Visual observation, image and/or video capturing, and/or image and/or video processing can be used to study, characterize, and record morphologic structures of the meibomian glands. For example, dysfunctional meibomian glands (e.g., stagnation of the gland complexes) can increase pressure in the gland, resulting in morphological changes. These changes are reflected in the morphologic structure, which can be observed and recorded with the device 100. As noted herein, prior art techniques provide no or very limited photographic recording. Prior art techniques also fail to provide a means to easily and effectively flip the eyelid while simultaneously transilluminating the eyelid with uniform, diffuse EMR that is within the therapeutic window. Embodiments of the device 100, however, can be used to easily and effectively flip the eyelid, simultaneously transilluminating the eyelid with uniform, diffuse EMR that is within the therapeutic window, and record images and/or video of the morphologic structure. With image and video processing, the device 100 can provide distilled information about morphological changes that can be quickly assessed for accurate diagnosis and treatment.

For example, the device 100 can be used to examine eyelid tissue by positioning the device 100 so that the light source 102 is adjacent an eyelid. A user can exert pressure to the eyelid through a movement of the device 100 towards the eyelid in an upward motion. This can cause the eyelid to be everted over at least a portion of the light emission region 106. The device 100 can generate the EMR having a uniform, diffuse electromagnetic transmission spanning the range from 650 nm to 2500 nm or an intermediate range within the range from 650 nm to 2500 nm. The detector unit 108 can detect interactions of the EMR with tissue and associated structures of the eyelid. The tissue and associated structures of the eyelid can include the meibomian glands. A user can identify morphologic structures of and/or morphological changes in the tissue and associated structures of the eyelid. This can include generating an image and/or a video of the tissue and associated structures of the eyelid via the computer 116. In addition, the computer 116 can be used to record morphologic structures of and/or morphological changes in the tissue and associated structures of the eyelid via the generated image and/or video.

It should be understood that modifications to the embodiments disclosed herein can be made to meet a particular set of design criteria. For instance, the number of or configuration of detector units 108, light sources 102, computers 116, communication networks 114 and/or other components or parameters may be used to meet a particular objective. In addition, any of the embodiments of the device 100 disclosed herein can be connected to other embodiments of the device 100 to generate a desired device 100 configuration.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

Therefore, it is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of apparatuses and methods of making and using the same have been discussed and illustrated herein, it is to be distinctly understood that the invention is not limited thereto but may be otherwise variously embodied and practiced within the scope of the following claims.

It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement. Thus, while certain exemplary embodiments of the device 100 have been shown and described above, it is to be distinctly understood that the invention is not limited thereto but may be otherwise variously embodied and practiced within the scope of the following claims.

What is claimed is:

1. A diagnostic tool, comprising:
at least one light source configured to generate electromagnetic radiation (EMR) within a range from 650 nm to 2500 nm, the EMR being emitted through a light emission region;
a detector unit configured to detect interactions of the EMR with tissue;
wherein the light emission region is configured as a diffuser to cause the EMR to be a uniform, diffuse electromagnetic transmission spanning the range from 650 nm to 2500 nm or an intermediate range within the range from 650 nm to 2500 nm.

2. The diagnostic tool recited in claim 1, wherein the light emission region is configured to cause the EMR to be a uniform, diffuse electromagnetic transmission spanning a range from 650 nm to 1870 nm or an intermediate range within the range from 650 nm to 1870 nm.

3. The diagnostic tool recited in claim 1, wherein the at least one light source comprises a light emitting diode (LED).

4. The diagnostic tool recited in claim 1, wherein the at least one light source comprises a plurality of light sources.

5. The diagnostic tool recited in claim 4, wherein the plurality of light sources comprises:
a first light source configured to generate EMR within a first predetermined band of wavelengths;
a second light source configured to generate EMR within a second predetermined band of wavelengths; and
a third light source configured to generate EMR within a third predetermined band of wavelengths.

6. The diagnostic tool recited in claim 5, wherein:
the first predetermined band of wavelengths is different from the second predetermined band of wavelengths and the third predetermined band of wavelengths;
the second predetermined band of wavelengths is different from the first predetermined band of wavelengths and the third predetermined band of wavelengths; and
the third predetermined band of wavelengths is different from the second predetermined band of wavelengths and the first predetermined band of wavelengths.

7. The diagnostic tool recited in claim 5, wherein:
the first predetermined band of wavelengths is less than the second predetermined band of wavelengths; and
the second predetermined band of wavelengths is less than the third predetermined band of wavelengths.

8. The diagnostic tool recited in claim 5, wherein:
the first predetermined band of wavelengths is within a range from 650 nm to 950 nm;
the second predetermined band of wavelengths is within a range from 1100 nm to 1350 nm;
the third predetermined band of wavelengths is within a range from 1600 nm to 1870 nm; and
the light emission region is configured to cause the EMR to be a uniform, diffuse electromagnetic transmission spanning a range from 650 nm to 1870 nm or an intermediate range within the range from 650 nm to 1870 nm.

9. The diagnostic tool recited in claim 1, wherein the light emission region includes a lens comprising glass, plastic, polymer, ceramic, and/or epoxy.

10. The diagnostic tool recited in claim 9, wherein lens is configured as a diffuser.

11. The diagnostic tool recited in claim 1, further comprising a controller configured to control the intensity of the EMR being emitted from the at least one light source.

12. The diagnostic tool recited in claim 11, wherein the controller is a variable resistor rheostat.

13. The diagnostic tool recited in claim 4, further comprising a controller configured to control the intensity of the EMR being emitted from each light source individually.

14. The diagnostic tool recited in claim 4, further comprising a controller configured to control the intensity of the EMR being emitted from each light source in unison.

15. A diagnostic tool, comprising:
a housing configured as a hand-held unit for placement adjacent tissue to be analyzed via spectroscopy;
a light emission region formed in a portion of the housing;
at least one light source configured to generate electromagnetic radiation (EMR) within a range from 650 nm to 2500 nm, the EMR being emitted through the light emission region;
a detector unit configured to detect interactions of the EMR with the tissue;
a power source configured to facilitate transfer of electrical current or electrical voltage from an electrical power supply;
wherein the light emission region is configured as a diffuser to cause the EMR to be a uniform, diffuse electromagnetic transmission spanning the range from 650 nm to 2500 nm or an intermediate range within the range from 650 nm to 2500 nm.

16. The diagnostic tool recited in claim 15, wherein the power supply is a battery unit.

17. A diagnostic system, comprising:
a diagnostic tool, comprising:
at least one light source configured to generate electromagnetic radiation (EMR) within a range from 650 nm to 2500 nm, the EMR being emitted through a light emission region; and
a detector unit configured to detect interactions of the EMR with tissue and generate detection data;
wherein the light emission region is configured as a diffuser to cause the EMR to be a uniform, diffuse electromagnetic transmission spanning the range from 650 nm to 2500 nm or an intermediate range within the range from 650 nm to 2500 nm;
a computer in communication with the diagnostic device, the computer configured to receive the detection data and generate an image and/or a video that is representative of the detection data.

18. The diagnostic system recited in claim 17, wherein the computer is configured to analyze the detection data via spectroscopy.

19. The diagnostic system recited in claim 17, wherein the computer is configured to generate a user interface programmed to facilitate image or video processing, enhancing, analysis, and/or storage of the image and/or the video.

20. The diagnostic system recited in claim 17, wherein the computer is configured to generate text superimposed over the image and/or the video, the text comprising dimensions of the tissue and/or structures associated with the tissue.

21. A method of examining eyelid tissue, the method comprising:
placing a light source adjacent an eyelid and applying pressure to the eyelid via exertion of pressure to the eyelid through a movement of the light source, the exertion of pressure causing the eyelid to be everted over at least a portion of the light source;
generating electromagnetic radiation (EMR) within a range from 650 nm to 2500 nm, the EMR being a uniform, diffuse electromagnetic transmission spanning the range from 650 nm to 2500 nm or an intermediate range within the range from 650 nm to 2500 nm; and
detecting interactions of the EMR with tissue and associated structures of the eyelid.

22. The method recited in claim 21, wherein the tissue and associated structures of the eyelid comprise meibomian glands.

23. The method recited in claim 21, further comprising identifying morphologic structures of the tissue and associated structures of the eyelid.

24. The method recited in claim 23, further comprising identifying morphological changes in the tissue and associated structures of the eyelid.

25. The method recited in claim 21, further comprising generating an image and/or a video of the tissue and associated structures of the eyelid.

26. The method recited in claim 25, further comprising recording morphologic structures of the tissue and associated structures of the eyelid via the image and/or the video.

27. The method recited in claim 26, further comprising recording morphological changes in the tissue and associated structures of the eyelid via the image and/or the video.
